(19) United States
(12) Patent Application Publication
(10) Pub. No.: US 2014/0273143 A1
(43) Pub. Date: Sep. 18, 2014

(54) Methods, Systems, and Culture Medium for Production of Dechlorinating Microorganisms

(71) Applicants: Rosa Krajmalnik-Brown, Chandler, AZ (US); Cesar Torres, Tempe, AZ (US); Anca Delgado, Tempe, AZ (US); Sudeep Popat, Chandler, AZ (US); Devyn Fajardo-Williams, Mesa, AZ (US)

(72) Inventors: Rosa Krajmalnik-Brown, Chandler, AZ (US); Cesar Torres, Tempe, AZ (US); Anca Delgado, Tempe, AZ (US); Sudeep Popat, Chandler, AZ (US); Devyn Fajardo-Williams, Mesa, AZ (US)

(73) Assignee: Arizona Board of Regents on behalf of Arizona State University, Scottsdale, AZ (US)

(21) Appl. No.: 14/204,058

(22) Filed: Mar. 11, 2014

Related U.S. Application Data
(60) Provisional application No. 61/784,520, filed on Mar. 14, 2013.

(51) Int. Cl. C12P 5/02

(52) U.S. Cl. CPC C12P 5/026
USPC 435/167; 435/293.1; 435/253.6

(57) ABSTRACT

Methods, systems, and compositions for growing high density cultures of dechlorinating microorganisms, such as the bacteria Dehalococcoides. Dechlorinating cultures are grown in continuous flow stirred-tank reactors at short hydraulic retention time, resulting in improved batch production. For some cultures, a culture medium including chlorine containing compounds, bicarbonate, and HEPES utilized.

### CROSS-REFERENCE TO RELATED APPLICATIONS

This application claims the benefit of U.S. provisional Application No. 61/784,033 filed on Mar. 14, 2013, which is incorporated by reference herein in its entirety.

### BACKGROUND OF THE INVENTION

The chlorinated organic solvents trichloroethene (TCE) and perchloroethene (PCE) are worldwide contaminants of soil and groundwater. In the United States, at least 60% of the National Priorities List (NPL) Superfund sites at least 17% of groundwater sources have detectable levels of chlorinated solvents, including TCE and PCE. The presence and persistence of chlorinated solvents in the environment is a major threat to public health and economic activities. Currently, biological reduction by members of the bacterial genus Dehalococcoides is a common and cost-effective avenue for remediation of sites contaminated with TCE and PCE. Dehalococcoides are the only microorganisms identified to date that can reductively dechlorinate PCE and TCE to ethene, the non-toxic end product, with transient production of cis-dichloroethene (cis-DCE), as the most common DCE isomer, and vinyl chloride (VC), because of this interest for growth and application of Dehalococcoides related remediating techniques has increased in the past 15 years.

Bioremediation practitioners employ bioaugmentation with Dehalococcoides-containing cultures following or concomitantly with biostimulation to achieve high and sustainable rates of chloroethene dechlorination. TCE- and PCE-dechlorinating cultures for bioaugmentation are commonly produced under batch-fed conditions to allow for a tight control on maintaining anaerobic conditions. To achieve high densities of Dehalococcoides, these cultures are commonly fed with high concentrations (mM range) of chloroethenes. However, in batch systems, self or competitive inhibition on dechlorination, and toxicity on Dehalococcoides and other community members, prevent feeding TCE or PCE in high concentrations. Therefore, batch production of bacteria cultures currently results in an inherently inefficient slow process.

Thus, improvements in methods and systems for production of Dehalococcoides that provide for coupling growth to fast rates of TCE or PCE dechlorination in a continuous flow stirred-tank are desirable.

### SUMMARY OF THE INVENTION

The embodiments described herein relate to methods and systems for continuous production of dechlorinating microorganisms with rapid rates of TCE or PCE dechlorination in continuous-flow stirred tank reactors (CSTRs).

In an embodiment, a method for growing dechlorinating microorganisms, including providing a culture medium including chlorinated compounds to a CSTR, flowing the culture medium at a specific hydraulic retention time, and inoculating the culture medium with at least one microorganism cell adapted to dechlorinate the chlorinated compounds. The method further includes culturing the at least one adapted bacterial cell in the presence of about 5 mM bicarbonate and HEPES, and converting the chlorine containing compounds to ethene.

In an embodiment, a system for growing dechlorinating microorganisms including a CSTR, a culture medium which includes chlorinated compounds, about 5 mM bicarbonate, and HEPES. The system also includes at least one bacterial cell adapted to dechlorinate the chlorinated compounds, a peristaltic pump to feed the culture medium into the CSTR, and an effluent collection bottle.

In an embodiment, a composition for selectively growing high-density cultures of Dehalococcoides in a selected culture medium. The culture medium including chlorinated compounds, about 2.5 mM to 7.5 mM bicarbonate, and about 10 mM to 25 mM HEPES.

These and other aspects of the invention will be apparent upon reference to the following detailed description and figures. To that end, any patent and other documents cited herein are hereby incorporated by reference in their entirety.

### BRIEF DESCRIPTION OF THE DRAWINGS

FIG. 1. A schematic and image of a system for production of Dehalococcoides illustrating the continuous flow stirred-tank reactor.

FIG. 2. Dechlorination of 1 mM TCE and 2 mM TCE influent and the corresponding percent ethene conversion in a CSTR operated at a 3-d HRT (hydraulic retention time). The percent ethene conversion at each time point was calculated using Equation 1. Light gray shaded areas illustrate periods of batch operation and a dashed line represents the start of the 2 mM TCE continuous feed.

FIG. 3. Microbial populations abundance in a 3-d HRT CSTR determined by qPCR at time 0 (no-fill bars), 1 mM TCE pseudo steady-state (light-filled bars), and 2 mM TCE pseudo steady-state (dark-filled bars). (A) Log concentrations of Dehalococcoides (DHC), Geobacteraceae (GEO), FTHFS (FTH), and Archaea (ARC). (B) Log concentrations of Dehalococcoides functionally-defined reductive dehalogenase genes, tceA, vcrA, and bvcA. All error bars show standard deviations of replicate samples (time 0, n=2; 1 mM TCE, n=4; 2 mM TCE, n=3) and analytical qPCR reactions (time 0, n=6; 1 mM TCE, n=12; 2 mM TCE, n=9).

FIG. 4. Experimental time-course measurements to determine the maximum rate of conversion, Rmax, for the culture produced in a 3-dHRT CSTR fed with an influent containing (A) 1 mM TCE and (B) 2 mM TCE. 0.5 mmol L⁻¹ TCE, cis-DCE, or VC was added to the effluent culture in serum batch bottles in separate experiments. The production rate of the lesser chlorinated products, cis-DCE, VC, or ethene, was measured over short periods (5 hours or less) to minimize microbial growth. The points are experimental measurements and the lines are linear fits of the experimental data.

FIG. 5. Viability and performance of DehaloR² culture produced in a CSTR after storage at 4° C. for 7 months and 15 months. Dechlorination of TCE to ethene was assessed by transferring 10 mL refrigerated culture into 90 mL reduced anaerobic mineral medium amended with TCE and electron donors. The error bars are standard deviations of triplicate cultures.

### DETAILED DESCRIPTION OF THE INVENTION

Embodiments described herein relate to an optimized protocol for the coupling of dechlorinating microorganism growth to rapid rates of TCE or PCE dechlorination. The utilization of this method allows for the successful growth of a dechlorinating bacterial-containing culture in a CSTR at short hydraulic retention time (HRT) using TCE as an electron acceptor. This method has been demonstrated to yield a sustained dechlorination of TCE to ≧97% ethene, coupled to the production of 10¹² Dehalococcoides cells per L culture.

Despite achieving high-cell densities, batch production of Dehalococcoides cultures is a slow process. To increase production time, a continuous-flow stirred tank reactor (CSTR) provides for more efficient and controlled cellular growth. In the CSTR, toxicity to microbial community members can be minimized by continuously maintaining low concentrations of TCE or PCE. These low concentrations enable feeding higher concentrations of chlorinated solvents than in a batch reactor, thus achieving a higher concentration of Dehalococcoides cells. Despite these potential advantages, chlorinated ethene CSTR studies are limited and with little or no success in achieving sustainable growth of completely dechlorinating cultures (a summary of previous CSTR studies is presented in Table 1).

TABLE 1
Summary of key parameters and microbial inocula employed in chlorinated ethenes
CSTR studies.

| | | | | | Major | |
|---|---|---|---|---|---|---|
| Chlorinated | e⁻ donor and | Undefined | | HRT | reduced | Inoculum |
| ethene | C source | nutrients | Buffer | (d) | product | culture |
| 1 mM TCE | 7.5 mM | | 15 mM | 3 | Ethene | DehaloR² |
| | lactate and 15 mM | | HEPES and | | | dechlorination |
| | methanol | | 5 mM | | | culture |
| | | | HCO₃⁻ | | | converting |
| 2 mM TCE | 10 mM lactate | | 20 mM | 3 | Ethene | TCE to ethene |
| | and 15 mM | | HEPES and | | | (this study) |
| | methanol | | 5 mM | | | |
| | | | HCO₃⁻ | | | |
| 1.12 mM | 4.3 mM | 0.02 g L⁻¹ | 35 mM | 50-55 | Ethene | Point Mugu |
| PCE | lactate | yeast | Na₂CO₃ and | | | (PM) |
| | | extract | 6 mM | | | dechlorinating |
| | | | K₂HPO₄ | | | culture |
| | | | | | | converting |
| | | | | | | PCE to ethene |
| 7.4 mM TCE | 25.6 mM | | CO₃²⁻ | 5.9-25.3 | cis-DCE | Evanite (EV) |
| | lactate | | | | | subculture |
| | | | | | | converting |
| | | | | | | PCE to cis- |
| | | | | | | DCE |
| 0.52 mM | 52 mM | 0.2 g L⁻¹ | 90 mM | 11 | VC | Methanol/PCE |
| PCE | methanol, 20 mM | yeast | HCO₃⁻ | 5.8 | cis-DCE | enrichment |
| | pyruvate | extract, 1% | | 2.9 | cis-DCE | culture |
| | or 80% | filter- | | | | converting |
| | H₂/20% CO₂, | sterilized | | | | PCE to VC and |
| | and 2 mM | cell | | | | ethene |
| | acetate | extracts or | | | | |
| | | spent | | | | |
| | | medium | | | | |
| ≦50 mM | 45 mM lactate | | 10 mM | ~2 | cis-DCE | Co-culture of |
| PCE | | | (NH₄)H₂PO₄ | | | Desulfitobacterium |
| (nominal) | | | and 20% | | | frappieri |
| | | | CO₂ | | | TCE1 and |
| | | | | | | Desulfovibrio |
| | | | | | | sp. strain |
| | | | | | | SULF1 |
| 0.2 g PCE in | 10 mM | 0.2 g L⁻¹ | 10 mM | 3 | cis-DCE | Methanol/ |
| hexadecane | formate | yeast | HCO₃⁻ | | | PCE enrichment |
| NAPL | | extract | | | | culture |
| | | | | | | converting |
| | | | | | | PCE to VC and |
| | | | | | | ethene |
| 0.98 mM | 1.7 mM | 0.02 g L⁻¹ | 14 mM | 36 | Ethene | Dechlorinating |
| PCE | benzoate | yeast | Na₂CO₃ and | | | source culture |
| | | extract | 3 mM | | | converting |
| | | | K₂HPO₄ | | | PCE to ethene |

The inventors believe that there are two major difficulties that may impede culturing of dechlorinating bioaugmentation cultures in a CSTR. First, inhibition of bacterial growth may occur due to toxicity of the chlorine containing compounds. For growth of dechlorinating cultures to occur, a high enough concentration of chlorinated solvents must be fed to attain high concentrations of Dehalococcoides. Yet, very high removal of TCE or PCE to ethene must occur to avoid cell growth inhibition (the effluent concentrations of chlorinated ethenes must be low). Secondly, there is stringent competition between Dehalococcoides and other community members for the obligate electron donor, hydrogen (H₂). In certain embodiments, methods and systems provide for the successful growth of Dehalococcoides-containing consortia in a CSTR at a 3-d HRT fed with 1 or 2 mM TCE. In certain embodiments, the methods and systems couple high-cell densities of Dehalococcoides to fast rates of dechlorination of TCE to ethene. Accordingly, the successful CSTR presented here is achieved with an optimized medium composition with low bicarbonate concentration, thus managing the microbial communities and achieving low effluent concentrations of chlorinated ethenes.

In certain embodiments, the methods and systems provide for growth of Dehalococcoides-containing cultures in CSTRs at short hydraulic retention time using feed with TCE as the electron acceptor. In an embodiment, the methods and system produce 10¹² Dehalococcoides cells per L culture at short HRTs. This outcome was made possible in part by using an optimized medium that minimizes the excessive proliferation of microorganisms competing with Dehalococcoides for H₂. In certain embodiments, the maximum conversion rates for the CSTR-[produced culture are 0.134±0.016, 0.055±0.018, and 0.017±0.00 mmol per L culture per hour, respectively, for TCE, cis-dichloroethene, and vinyl chloride. Depending on the site to be remediated, bioaugmentation can require hundreds to thousands of liters of bioaugmenting culture. For effective remediation, 10⁷ Dehalococcoides cells must be present in one liter groundwater. At a current price of up to $300 per liter culture, it is crucial to develop alternative methods to produce high-cell density Dehalococcoides inocula in order to achieve targeted and cost-effective contaminant removal. Using our carefully selected conditions in a CSTR, 10¹² Dehalococcoides cells per liter can be produced at short HRTs, thus minimizing costs in media components, reactor size and/or time of operation. Our CSTR approach provides a substantially increased production rate of high-cell density Dehalococcoides culture.

### Growth Conditions

These methods and systems allow for growth of Dehalococcoides containing cultures that convert the electron acceptor TCE to mostly ethene in a CSTR at an about 3-d HRT.

### Medium

The growth medium developed minimizes the proliferation of H₂ competitors in anaerobic cultures as it contains low bicarbonate (5 mM). Because a medium with only 5 mM bicarbonate would not suffice to buffer substantially all the protons produced from dechlorination and fermentation when producing high-density Dehalococcoides, buffering capacity was increased by supplementation with HEPES. HEPES is not substantially metabolized by any known microbe under anaerobic conditions. However, other buffers other than HEPES including but not limited to phosphate, MOPS, ammonium can be used when growing bioaugmentation cultures. Additionally, the medium used substantially enhances the growth of microbes beneficial to Dehalococcoides, by using the combination of lactate and methanol as electron donors at optimized concentrations.

In certain embodiments, the growth medium includes a certain concentration HEPES, e.g., in concentrations of about 10 mM to about 25 mM, about 15 mM to about 20 mM, about 25 mM, about 20 mM, about 15 mM, or about 10 mM.

In certain embodiments, the growth medium includes bicarbonate in concentrations of about 2.5 mM to about 7.5 mM, about 4.0 mM to about 6.0 mM, about 2.5 mM, about 5.0 mM, or about 7.5 mM.

In certain embodiments, the growth medium includes sodium DL-lactate in concentrations of about 5.0 mM to about 10.0 mM, about 10 mM, about 7.5 mM, or about 10.0 mM.

In certain embodiments, the growth medium includes methanol in concentrations of about 10 mM to about 25 mM, about 15 mM to about 20 mM, about 10 mM, about 15 mM, about 20 mM, or about 25 mM.

In certain embodiments, the growth medium includes TCE in concentrations of about 0.10 mM to about 4.0 mM, about 0.5 mM to about 3.0 mM, about 1.0 mM, about 2.0 mM, or about 3.0 mM.

In certain embodiments, the growth medium includes ATCC vitamin supplement in concentrations of about 2.5 mL/L to about 7.5 mL/L, about 4.0 mL/L to about 6.0 mL/L, about 4 mL/L, about 5 mL/L, or about 6 mL/L

In certain embodiments, the growth medium includes vitamin B₁₂ supplement in concentrations of about 250 μg/L to about 750 μg/L, about 400 μg/L to about 600 μg/L, about 400 μg/L, about 500 μg/L, or about 600 μg/L.

In certain embodiments, the growth medium includes resazurin in concentrations of about 0.10 μg/L to about 0.40 μg/L, about 0.20 μg/L to about 0.30 μg/L, about 0.20 μg/L, about 0.25 μg/L, or about 0.30 μg/L.

In certain embodiments, the growth medium includes L-cysteine in concentrations of about 0.10 mM to about 0.30 mM, about 0.15 mM, about 0.20 mM, about 0.25 mM, or about 0.30 mM.

In certain embodiments, the growth medium includes Na₂S×9 H₂O in concentrations of about 0.10 mM to about 0.30 mM, about 0.15 mM, about 0.20 mM, about 0.25 mM, or about 0.30 mM.

### Reactor

The CSTR is constructed using a glass bottle and stainless steel tubing. Viton tubing is also used in the peristaltic pump. A common trait of these materials is that they are impermeable to oxygen. This reactor design minimized possible inhibition of anaerobic microorganisms in the CSTR.

### Non-Limiting Working Example

### CSTR Design and Operation

To achieve this successful CSTR operation, we built upon data from prior CSTR runs in our laboratory (Table 2) with different operating conditions (TCE concentration, electron donor concentration, and HRT) in 30 mM bicarbonate (HCO3-)-buffered medium and a previous systematic study evaluating HCO3- as an electron acceptor in microbial dechlorination of TCE. In the HCO3- study, we saw that high HCO3- levels (i.e., 30 mM) increase the H₂ demand by stimulating homoacetogenesis and methanogenesis, two processes competing for H₂ and therefore, potentially limiting reductive dechlorination of chloroethenes.

TABLE 2
Experimental conditions tested the CSTR optimization.

| | | [Substrate]_{influent} | | | | |
|---|---|---|---|---|---|---|
| | | | | | HCO₃⁻/CO₂ | |
| | HRT | TCE | Lactate | Methanol | buffer | |
| Run | (d) | (mM) | (mM) | (mM) | (mM) | Notes |
| 1 | 4 | 3 | 10 | 12 | 30 | Ethene was the most prevalent |
| | | | | | | dechlorination end-product |
| | | | | | | throughout three HRTs; no |
| | | | | | | significant methanogenesis. |
| 2 | 2 | 4 | 10 | 12 | 30 | Ethene was the most prevalent |
| | | | | | | dechlorination end-product |
| | | | | | | throughout three HRTs; no |
| | | | | | | significant methanogenesis. |
| 3 | 8 | 8.37 | 15-20 | 12 | 30 | Conversion to mostly ethene |
| | | | | | | occurred initially, however TCE |
| | | | | | | accumulated after two HRTs |
| | | | | | | and performance did not |
| | | | | | | recovered; active |
| | | | | | | methanogenesis. |
| 4 | 4 | 8.37 | 20 | 12 | 30 | Conversion to ethene and VC |
| | | | | | | occurred within the first two |
| | | | | | | HRTs. cis-DCE accumulated |
| | | | | | | after four HRTs and |
| | | | | | | performance did not recover; |
| | | | | | | active methanogenesis. |
| 5 | 3 | 4 | 20 | 12 | 30 | TCE accumulated after four |
| | | | | | | HRTs and performance did not |
| | | | | | | recover. |
| 6 | 4 | 4 | 20 | 12 | 30 | Conversion to cis-DCE and VC |
| | | | | | | occurred operating for six |
| | | | | | | HRTs; active methanogenesis. |
| 7 | 3 | 1 | 7.5 | 15 | 5 (+20 mM | Conversion to mainly ethene |
| | | | | | HEPES) | was achieved and was |
| | | | | | | sustained. |
| 8 | 3 | 2 | 10 | 15 | 5 (+20 mM | Conversion to mainly ethene |
| | | | | | HEPES) | was achieved and was |
| | | | | | | sustained. |

A schematic and photograph showing the concept and actual continuous reactors are shown in FIG. 1. Each reactor consisted of a 0.65-L glass bottle sealed with a butyl rubber stopper and a screw cap. The stopper was perforated to fit the influent and effluent lines, and a gas sampling port containing a removable septum. The actual liquid and headspace operating volumes were 0.5 L and 0.1 L, respectively. Each reactor was stirred with a magnetic stirrer at 200 RPM, and was submerged in a water bath set at 30° C. Influent media was pumped from 5-L glass bottles containing 4 L of medium with a peristaltic pump to achieve a 3-d HRT. All lines and tubing used were ⅛″ diameter stainless steel or Viton material. The liquid sampling port was located before the effluent collection bottle. The effluent culture was collected into 1-L glass bottles equipped with 1-L gas Tedlar bags for gas collection.

### Inoculum Culture and Medium Composition

The culture employed for the CSTR studies was DehaloR², a TCE to ethene dechlorinating consortium containing Dehalococcoides and Geobacter. DehaloR² inoculum was grown in a CSTR fed with 3 mM TCE at a 4-d HRT (Table 2, Run 1) and stored at 4° C. for 15 months prior to inoculating the bioreactors presented herein. 0.5 L DehaloR² culture (100% vol/vol) per reactor was inoculated on day 0. Trace concentrations of cis-DCE and VC were present in this culture during storage; therefore, we added 2 mM lactate and kept the reactors in batch mode for ˜4 days to reduce the chlorinated ethenes to ethene before proceeding to continuous operation.

We prepared reduced anaerobic mineral medium containing 1 mM TCE (aqueous concentration), 7.5 mM sodium DL-lactate, 15 mM methanol, 15 mM 4-(2-hydroxyethyl)-1-piperazineethanesulfonic acid (HEPES), 5 mM NaHCO3, 5 mL L-1 ATCC vitamin supplement, 500 μg L-1 vitamin B12, 0.25 μg L-1 resazurin, 0.2 mM L-cysteine, and 0.2 mM Na₂S×9 H₂O. In the medium with 2 mM TCE, the lactate and HEPES were increased to 10 mM and 20 mM, respectively, NaCl was decreased to 0.1 g L-1, and methanol was kept at 15 mM. The influent medium pH was adjusted to 7.5-7.8 with 10 N NaOH. The same base medium composition was used for previous CSTR runs presented in Table 1, except the noted differences summarized in the table. We first autoclaved the medium, boiled it under a stream of UHP N₂, and then added the reducing agents. To avoid fluctuations in TCE concentrations in the media bottles from changes in the liquid-headspace ratios during continuous operation, the bottles were fitted with collapsible 3-L gas Tedlar bags filled with UHP N₂.

### Chemical Analyses

We sampled gas from the headspace of the reactors to quantify TCE, DCE, VC, ethene, methane, and H₂ using Shimadzu gas chromatography instruments.

Conversion of the influent TCE in the reactors was obtained according to the mol balance equation below:

[TCE]ᵢₙ×Q_{liq}=[Ethenes]_{out gas}×Q_{gas}+[Ethenes]_{out liq}×Q_{liq} (Equation 1)

in which [TCE]=TCE aqueous concentration (mM), [Ethenes]=cumulative concentration of chlorinated ethenes (TCE, cis-DCE, VC), and ethene in the reactor and effluent (mM), and Q=flow rate (mL d⁻¹). The concentrations of TCE in the influent media and ethenes in the headspace of the reactors were quantified by GC-FID, while concentrations in the liquid were calculated using Henry's constants (K_{H}) for each compound:

[Compound]_{liq}=[Compound]_{gas}/K_{H} (Equation 2)

We obtained dimensionless Henry's constants (mM_{gas}/mM_{liq}) experimentally for the mineral medium used in this study for TCE (0.49), cis-DCE (0.17), VC (1.32), and ethene (9.00). Q_{gas} was the only parameter that could not be measured in our experiments; therefore, it was calculated from Equation 1.

### Microbial Ecology

We extracted total genomic DNA from pellets made with 1.5 mL liquid samples according to the protocol previously published. Quantitative real-time PCR (qPCR) assays were performed targeting the 16S rRNA genes of Dehalococcoides, Geobacteraceae, Eubacteria, and Archaea, and formyltetrahydrofolate synthase (FTHFS) (gene involved in the pathway for acetate production by homoacetogens). We also performed qPCR tracking the reductive dehalogenase genes of Dehalococcoides, tceA, vcrA, and bvcA, using the qPCR protocol, primers, probes, reagent concentrations, and PCR conditions detailed previously except each reductive dehalogenase gene was assayed separately.

### Conversion Rates and Long-Term Viability of CSTR-Grown Culture

Once pseudo steady-state was achieved for the 1 mM and 2 mM TCE continuous runs, we determined the maximum rates of conversion, Rₘₐₓ, for TCE, cis-DCE, and VC. We transferred 100 mL effluent culture to 160-mL serum glass bottles, and flushed for 20 min with UHP N₂ gas to remove any carry-over ethenes. Then, we provided a chlorinated electron acceptor (about 0.5 mmol L_{culture}⁻¹ of either TCE, cis-DCE, or VC), 5 mM lactate, 12 mM methanol, and 10 mL H₂ (4.1 mmol L_{culture}⁻¹). We measured the concentration of dechlorination products formed over short time intervals (five hours or less) in order to minimize increases in dechlorinating populations. qPCR tracking the Dehalococcoides 16S rRNA gene was employed to confirm that these bacteria had not grown significantly throughout the course of the tests. All Rₘₐₓ values were determined from at least triplicate experiments.

The culture produced in the CSTR from Runs 1-2 and 7-8 in Table 1 was stored in a 4° C. refrigerator and routinely monitored for activity at few different time intervals. Viability experiments consisted of transferring 10 mL stored culture to 160-mL serum bottles containing 90 mL anaerobic medium (10% inoculum vol/vol), adding 0.5-1 mmol L_{culture}⁻¹ TCE, 5 mM lactate, and 12 mM methanol, and monitoring TCE dechlorination to ethene in time course experiments.

Dechlorination Performance of a Dehalococcoides-Containing Culture in a 3-d HRT CSTR Fed with 1 mM and 2 mM TCE

FIG. 2 shows the performance of one CSTR when fed 1 mM TCE at a 3-d HRT. cis-DCE and VC initially accumulated in the bioreactors within the first two HRTs; however, by day 11, ethene became the prevalent dechlorination end-product, and >90% conversion of TCE to ethene was observed thereafter. When the influent was 2 mM TCE, the bioreactors exhibited the same conversion trends as when initially fed with 1 mM TCE (FIG. 2). For the first several HRTs, cis-DCE and VC were the main dechlorination products. Conversion to mostly ethene was achieved by day 65 but performance declined shortly after (FIG. 2). We believe this decline was due to an oxygen leak into the reactor from a damaged influent pump tubing. Once the tubing was replaced, the reactor recovered and a pseudo steady-state with greater than 93% conversion to ethene was also achieved by day 94 with 2 mM TCE influent concentration, and was sustained for 9 subsequent HRTs (FIG. 2).

### Growth of Dehalococcoides and Enrichment of Efficient Microbial Dechlorinating Communities

We monitored the growth of Dehalococcoides every HRT until pseudo steady-state was achieved. FIG. 3A shows the initial concentration of Dehalococcoides and the average steady-state abundances of 1.31×10¹² and 1.57×10¹² cells L_{culture}⁻¹ when continuously feeding 1 or 2 mM TCE, respectively, at a 3-d HRT.

As revealed in Table 1, our study was performed using a defined medium, with the implications that the microbial community enriched under the selective pressure of a 3-d HRT and the medium composition used was self-sustainable, and provided any undefined nutrients for the optimal growth of Dehalococcoides-containing cultures.

In terms of Dehalococcoides diversity/composition, the CSTR-grown culture contained the three previously identified reductive dehalogenase genes, tceA, vcrA, and bvcA. FIG. 3B shows that concentrations of the three reductive dehalogenase genes increased during operation, reaching highest levels during the 2 mM TCE pseudo-steady state with abundances of 10¹¹ copies L⁻¹ for tceA and vcrA, and 10⁸ copies L⁻¹ for bvcA.

Besides Dehalococcoides, DehaloR² culture contains one other identified dechlorinating genus, Geobacter, which only partially reduces TCE to cis-DCE. FIG. 3A shows Geobacteraceae 16S rRNA genes increasing throughout the two operating conditions. Data on growth of TCE/PCE-reducing Geobacter (lovleyi) in a pure culture or in a consortium are absent from the literature; however, the densities obtained for Geobacteraceae in our CSTRs are also on the high end compared to those in batch-fed mixed dechlorinating cultures. Our study shows that Dehalococcoides and Geobacter growth correlated to good performance for TCE dechlorination in continuous-flow reactors. Moreover, Geobacter has been documented to provide Dehalococcoides with required corrinoids for dechlorinating activity and cellular growth and therefore may be a desired partner in Dehalococcoides-containing cultures.

As shown in Table 1, in previous CSTR studies, the main reduced end-product of dechlorination of TCE and PCE was cis-DCE. This is suggesting that Dehalococcoides respiring cis-DCE or VC to ethene were inhibited by high concentrations of chlorinated solvents, washed out, or outcompeted by other microbes. Our study is the first to document conversion to mostly ethene in a CSTR at a 3-d HRT (Table 1). The high abundances of dechlorinators (Dehalococcoides and Geobacteraceae) obtained in our CSTRs clearly support the opportunity for their efficient growth in continuous reactors at short HRTs. Additionally, the community data on methanogens and homoacetogens in conjunction with the CSTR dechlorination performance to ethene support the idea that competing sinks for H₂ are minimized using our medium composition, thus allowing H₂ to be used for dechlorination.

TABLE 3
Maximum conversion rate (Rₘₐₓ) of chloroethenes by DehaloR² culture
produced in a CSTR fed with 1 mM TCE and 2 mM TCE influent
concentrations. The Rₘₐₓ values are averages with standard
deviations of at least triplicate experiments.

| | Rₘₐₓ | | |
|---|---|---|---|
| | (mmol L_{culture}⁻¹ h⁻¹) | | |
| [TCE]ᵢₙ | TCE | cis-DCE | VC |
| 1 mM | 0.044 | 0.023 | 0.007 |
| | (±0.004) | (±0.002) | (±0.001) |
| 2 mM | 0.134 | 0.055 | 0.017 |
| | (±0.016) | (±0.018) | (±0.007) |

### Dechlorination Kinetics of the Culture Produced in a CSTR

Table 3 summarizes the maximum conversion rates, Rₘₐₓ, at pseudo-steady state obtained from the culture produced in the CSTR grown with the two concentrations of TCE. Experimental data for the values in Table 2 are shown in FIG. 4. With the culture produced when continuously feeding 2 mM TCE, we obtained an Rₘₐₓ value for TCE dechlorination to ethene of 0.157 (±0.010) mmol CF released L_{culture}⁻¹ h⁻¹. This rate surpasses the previously reported batch-grown DehaloR² maximum rate of 0.038 mmol Cl⁻ released L_{culture}⁻¹ h⁻¹ (or 0.92 mmol Cl⁻ released L_{culture}⁻¹ d⁻¹), which was obtained by feeding a total of 3 mmol L_{culture}⁻¹ TCE in three consecutive additions of 1 mmol L⁻¹. As seen in Table 3, for the culture produced in this study, TCE to cis-DCE and cis-DCE to VC Rₘₐₓ are approximately four times higher than those reported by SDC-9 culture 0.036 and 0.015 mmol L_{culture}⁻¹ h⁻¹ respectively), while VC to ethene rates of DehaloR² measured here are lower than those of SDC-9 by a factor of two 0.039 mmol L_{culture}⁻¹ h⁻¹).

The lower Rₘₐₓ for VC compared to TCE and cis-DCE (Table 3) imply that the limiting step in the CSTRs was dechlorination of VC. VC to ethene is commonly the slowest dechlorination step, which might explain some of the rate differences between VC and TCE and cis-DCE dechlorination. Another factor we identified that could have led to lower apparent rates for VC dechlorination is the poorer gas-liquid transfer properties of VC, given its higher Henry's constant. In abiotic experiments using our medium composition (data not shown), we determined that 0.5 mmol L⁻¹ VC added as gas did not equilibrate between the liquid and gas within the time of the Rₘₐₓ experiments (5 hours or less). The slower dissolution of VC into the medium might have limited its bioavailability; hence, the reported values for VC in Table 2 are at least the minimum Rₘₐₓ for this electron acceptor, but the rates were likely higher as we did not observe significant VC accumulation during reactor operation (FIG. 2).

Culture Viability after Prolonged Storage

An advantage from producing dense microbial cultures containing Dehalococcoides is that they can be cultured before usage for laboratory or field applications, and stored for prolonged periods without significant loss in activity. The culture initially produced in our CSTR (Run 1, Table 2) was stored for prolonged periods at 4° C. FIG. 5A shows that complete dechlorination of ˜0.7 mmol L_{culture}⁻¹ TCE occurred in 6 days after the culture had been stored in a refrigerator for seven months. After 15 months, the same concentration of TCE was reduced to 80% ethene in 15 days (FIG. 5B), implying that, while some loss of activity will occur (as expected, due to cell decay), these cultures maintain good dechlorinating activity profiles when the appropriate conditions are provided.

The claims are not intended to be limited to the materials and methods described above, nor to the embodiments and examples described herein.

1. A method for dechlorination comprising:
providing a culture medium including chlorinated compounds, to a continuous flow stirred-tank reactor;
flowing the culture medium at achieve a specific hydraulic retention time;
inoculating the culture medium with a culture adapted to dechlorinate the chlorinated compounds;
culturing the culture in the presence of about 5 mM bicarbonate and HEPES; and
converting the chlorinated compounds to ethene.

2. The method of claim 1, further comprising culturing the culture in the presence lactate and methanol.

3. The method of claim 1, wherein said culture contains at least one Dehalococcoides cell.

4. The method of claim 1, wherein said culture medium is provided to an anaerobic environment within the continuous flow stirred-tank reactor.

5. The method of claim 1, wherein said culture medium includes trichloroethene or perchloroethene.

6. The method of claim 1, further comprising minimizing a proliferation of H₂ competitors in the culture medium.

7. The method of claim 1, wherein flowing the culture medium at a specific hydraulic retention time (HRT) comprises 3 days.

8. A system for dechlorination comprising:
a continuous flow stirred-tank reactor;
a culture medium in fluid communication with the continuous flow stirred-tank reactor wherein the culture medium comprises,
chlorinated compounds;
about 5 mM bicarbonate; and
HEPES;
a culture adapted to dechlorinate the chlorinated compounds;
a peristaltic pump in fluid communication with both the continuous flow stirred-tank reactor and the culture medium; and
an effluent collection bottle in fluid communication with the continuous flow stirred-tank reactor.

9. The system of claim 8, wherein the culture medium further comprises lactate and methanol.

10. The system of claim 8, wherein the culture adapted to dechlorinate the chlorine containing compounds comprises at least one Dehalococcoides cell.

11. The system of claim 8, wherein the continuous flow stirred-tank reactor comprises an anaerobic environment within the continuous flow stirred-tank reactor.

12. The system of claim 8, wherein the chlorine containing compounds comprise at least one of trichloroethene and perchloroethene.

13. The system of claim 8, wherein the culture medium flows through the continuous flow stirred-tank reactor at a hydraulic retention time (HRT) of about 3 days.

14. A composition for selectively growing high density cultures of Dehalococcoides comprising:
a culture medium including,
chlorinated compounds;
about 2.5 mM to about 7.5 mM bicarbonate; and
about 10 mM to about 25 mM HEPES.

15. The composition for selectively growing high density cultures of Dehalococcoides of claim 14, wherein the chlorinated compounds comprise about 0.10 mM to about 4.0 mM TCE.

16. The composition for selectively growing high density cultures of Dehalococcoides of claim 14, further comprising lactate and methanol.

17. The composition for selectively growing high density cultures of Dehalococcoides of claim 14, further comprising about 5.0 mM to about 10.0 mM sodium DL-lactate.

18. The composition for selectively growing high density cultures of Dehalococcoides of claim 14, further comprising about 10 mM to about 25 mM methanol.

19. The composition for selectively growing high density cultures of Dehalococcoides of claim 14, further comprising:
about 2.5 mL/L to about 7.5 mL/L ATCC vitamin supplement; and
about 250 μg/L to about 750 μg/L vitamin B₁₂.

20. The composition for selectively growing high density cultures of Dehalococcoides of claim 14, further comprising:
about 0.10 μg/L to about 0.40 μg/L resazurin;
about 0.1 mM to about 0.3 mM L-cysteine; and
about 0.1 mM to about 0.3 mM Na₂S×9 H₂O.
